(19) United States
(12) Patent Application Publication
(10) Pub. No.: US 2008/0171372 A1
(43) Pub. Date: Jul. 17, 2008

(54) PROCESS FOR DYNAMIC CONCENTRATION AND SEPARATION OF BACTERIA

(76) Inventors: Andrey V. Sokolov, Bolingbrook, IL (US); Igor Aronson, Darien, IL (US); Wai-Kwong Kwok, Downers Grove, IL (US)

Correspondence Address: JOAN PENNINGTON, 535 NORTH MICHIGAN AVENUE, UNIT 1804, CHICAGO, IL, 60611, US

(21) Appl. No.: 11/747,054

(22) Filed: May 10, 2007

Related U.S. Application Data
(60) Provisional application No. 60/845,378, filed on Sep. 18, 2006.

(51) Int. Cl. C12N 13/00; C12M 1/00

(52) U.S. Cl. 435/173.9; 435/283.1

(57) ABSTRACT

A method and apparatus for concentration and separation of bacteria in confined geometries such as in thin fluid films and in channels. The bacteria live in an environment of a specified level of pH, so that an increase or decrease of pH levels stimulates the bacteria to avoid areas of unfavorable pH and swim in the direction of the pH gradient. To create the gradient of pH an electric current is transmitted through a thin film or channel containing bacteria. The transmission of the current results in electrolysis in the vicinity of the electrodes and deviation of the pH levels from the bulk values. The bacteria swim away from the electrodes toward the center of the cell, where the pH level is more favorable.

This application claims the benefit of U.S. Provisional Application No. 60/845,378, filed on Sep. 18, 2006.

### CONTRACTUAL ORIGIN OF THE INVENTION

The United States Government has rights in this invention pursuant to Contract No. W-31-109-ENG-38 between the United States Government and The University of Chicago and/or pursuant to Contract No. DE-AC02-06CH11357 between the United States Government and UChicago Argonne, LLC representing Argonne National Laboratory.

### FIELD OF THE INVENTION

The present invention relates to a method for the concentration of bacteria. More specifically this invention relates to a process for the concentration and separation of swimming flagellated bacteria. Still more specifically this invention relates to a method of concentration and separation of bacteria in confined geometries such as thin fluid films and channels.

### DESCRIPTION OF THE RELATED ART

A need exists for an effective mechanism to separate and concentrate small quantities of live and dead bacteria in confined geometries, such as micro-channels and films.

Such a mechanism can be used for the purpose of express analysis, diagnostic, and identification of small bacterial samples, and separation sicken/live cells.

A principal aspect of the present invention is to provide a method and apparatus for concentration and separation of bacteria in confined geometries such as in thin fluid films and in narrow channels.

Other important aspects of the present invention are to provide such method and apparatus for concentration and separation of bacteria in confined geometries such as in thin fluid films and in channels, substantially without negative effect and that overcome some of the disadvantages of prior art arrangements.

### SUMMARY OF THE INVENTION

In brief, a method and apparatus for concentration and separation of bacteria. A gradient of pH is created using an electric current that is transmitted through a cell containing bacteria. The transmission of the current results in electrolysis in the vicinity of the electrodes and deviation of the pH levels from the bulk values. The bacteria swim away from the electrodes toward a center of the cell, where the pH level gradient is favorable for the bacteria.

In accordance with features of the invention, the bacteria live in an environment with a specific level of pH, so that an increase or decrease in pH levels stimulates the bacteria to avoid areas of unfavorable pH and to swim in the direction of the pH gradient. Since only living bacteria respond to the pH stimulation, by using this method one can separate living and dead cells or bacteria with different motility. The bacteria are contained in confined geometries, such as thin fluid films and narrow channels. The invention is suitable for flagellated bacteria such as E. coli, Bacillus subtilis, among many others, which relies on the ability of bacteria to swim toward areas of optimal pH level. This invention can be used in microfluidic diagnostic kits and miniature medical devices.

### BRIEF DESCRIPTION OF THE DRAWINGS

The present invention together with the above and other objects and advantages may best be understood from the following detailed description of the preferred embodiments of the invention illustrated in the drawings, wherein:

FIG. 1 is a schematic diagram representation of apparatus for concentration and separation of bacteria in confined geometries in accordance with the preferred embodiment;

FIG. 2 illustrates a bio-response of living bacteria contained in the apparatus of FIG. 1 to an applied electric current in accordance with the preferred embodiment;

FIG. 3 is a chart illustrating number of bacteria relative to the vertical axis and time in seconds relative to the horizontal axis for bacteria in the vicinity of electrode (first band) and at a distance of 300 μm (micro-meters) from electrode (second band) in response to an applied electric current in accordance with the preferred embodiment;

FIG. 4 is a schematic diagram representation of alternative apparatus for concentration and separation of bacteria in confined geometries in accordance with the preferred embodiment

### DETAILED DESCRIPTION OF THE PREFERRED EMBODIMENTS

In accordance with features of the preferred embodiments, apparatus and a new method are provided for concentration and separation of swimming flagellated bacteria, such as E. coli, Bacillus subtilis and many others in confined geometries, such as in thin fluid films and in channels. The method relies on the ability of bacteria to swim towards area of optimal pH level. The bacteria live in an environment of rather specific level pH; increase or decrease of pH stimulates living bacteria to avoid areas of unfavorable pH and swim in the direction of the pH gradient. The effect is similar to chemotaxitic response of bacteria on gradients of certain chemicals. To create a gradient of pH, an electric current is transmitted through a thin film containing bacteria. The transmission of the current results in electrolysis in the vicinity of the electrodes and deviation of the pH levels from the bulk values. The bacteria swim away from electrodes towards the center of the cell, where the pH level is favorable for bacteria. Since only living bacteria respond to the pH stimulation, using this method one can separate living and dead cells, or bacteria with different motility.

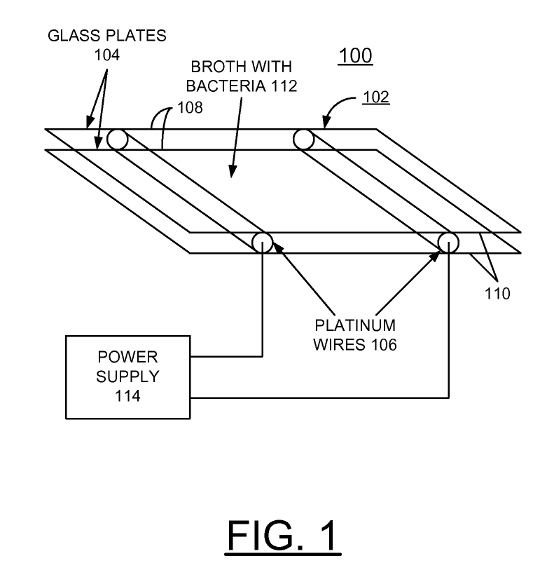

Having reference now to the drawings, FIG. 1 illustrates exemplary apparatus for concentration and separation of bacteria in confined geometries in accordance with the preferred embodiment and generally designated by the reference numeral 100.

Apparatus 100 includes an experimental cell generally designated by the reference numeral 102 for containing the bacteria. The experimental cell 102 includes a pair of glass plates 104 with a pair of spaced apart platinum wires 106 extending between opposite sides 108, 110 of the glass plates 104. A microchannel 112 containing a broth with the bacteria is defined between the glass plates 104 and the platinum wires 106. The platinum wires 106 are attached to a low voltage power supply source 114.

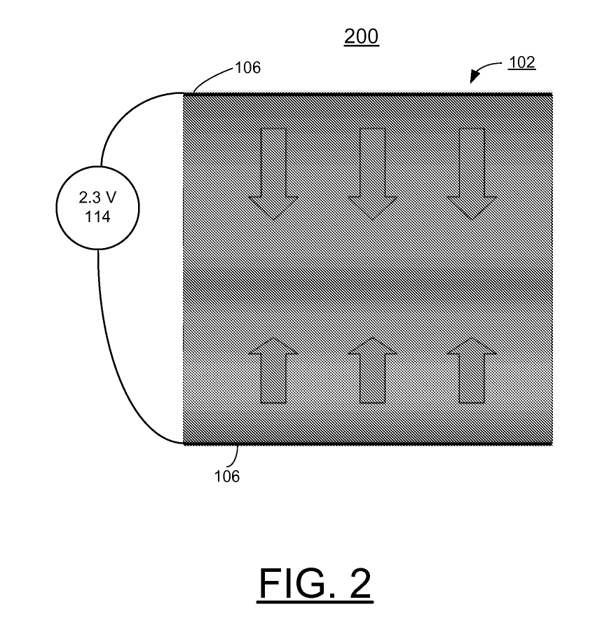

FIG. 2 shows a bio-response generally designated by the reference numeral 200 of bacteria contained in the apparatus 100 of FIG. 1 to an applied electric current in accordance with the preferred embodiment. As shown, the power supply has a voltage of 2.3 Volts to provide a total current of approximately 10 μA (10 micro-Amperes) causing a local change of pH level proximate to the platinum wire electrodes 106. The bacteria moves as indicated by multiple arrows or swims to escape the altered pH area near the electrodes 106 toward the center of the cell 102.

For example, flagellated bacteria, such as Bacillus subtilis, are about 5-10 μm (5-10 micro-meters) long and capable of swimming up to 20 μm/s (20 micro-meters per second). By transmitting electric current through the experimental cell microchannel 112, live bacteria is separated from dead bacteria. Different types of bacteria can be separated and identified. As illustrated in FIG. 2, the bacteria can be concentrated in a general area, such as within the central area of microchannel 112. Transmitting electric current through the experimental cell microchannel 112 can be used to manipulate living cell.

Experimental results have produced a sequence of microscope images illustrating effect of electric field on bacteria. For example, after 30 minutes, all living bacteria concentrate in the middle of the experimental cell, while the dead ones are left behind. Using this method the inventors succeeded in significantly increasing the concentration of the bacteria on a rather short time scale.

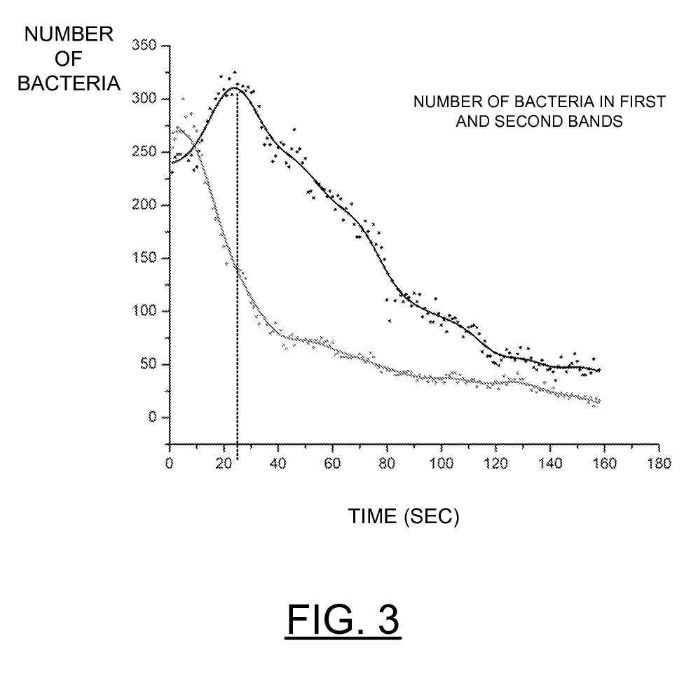

FIG. 3 is a chart illustrating the number of bacteria relative to the vertical axis and time in seconds relative to the horizontal axis for bacteria in the vicinity of electrode (first band) and at a distance of 300 μm (micro-meters) from electrode (second band) in response to an applied electric current in accordance with the preferred embodiment. Note that the two types of bacteria have different peaks of highest concentration with respect to time. The velocity of concentrated regions or avalanche is around an average velocity of the bacteria.

In brief, a significant advantage is that the method does not require either a large amount of bacteria or high-voltage or high electric power sources, can operate with very small quantities and in confined geometries. Concentrated bacteria can be individually counted and analyzed by an image processing technique. This method of the invention advantageously can be used for medical diagnostic and fast bioanalysis applications.

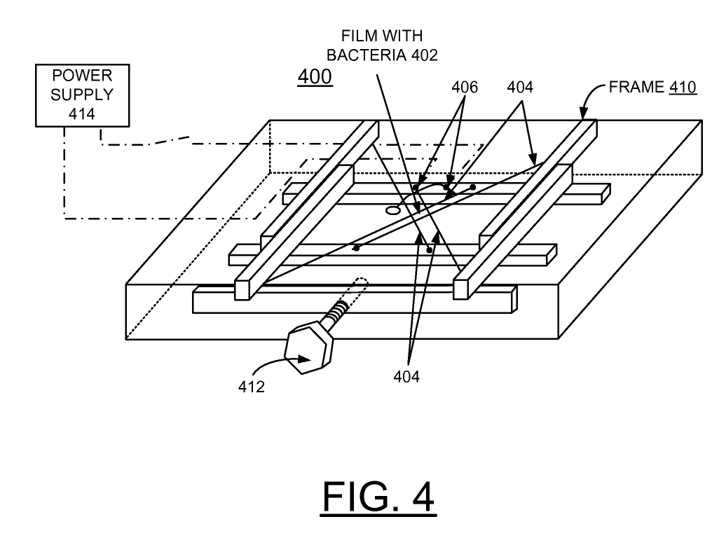

Referring now to FIG. 4, there is shown another apparatus for concentration and separation of bacteria in confined geometries in accordance with the preferred embodiment and generally designated by the reference numeral 400.

Apparatus 400 includes an experimental open film geometry generally designated by the reference numeral 402 for containing the bacteria. The experimental open film geometry 402 includes a plurality of supporting fibers 404, a pair of spaced apart platinum wires 406 and a pair of dielectric or glass fibers 408. The fluid film 402 containing the bacteria is stretched between these four fibers 404 by a frame structure generally designated by the reference numeral 410. Frame structure 410 includes a threaded connection 412 arranged to move frame arms to stretch the fluid film 402. The platinum wires 406 are attached to low voltage source 414.

While the present invention has been described with reference to the details of the embodiments of the invention shown in the drawing, these details are not intended to limit the scope of the invention as claimed in the appended claims.

What is claimed is:

1. A method for concentration and separation of bacteria comprising the steps of:
providing a cell containing the bacteria;
providing a pair of electrodes coupled to said cell containing the bacteria; and
creating a gradient of pH by transmitting an electric current through said cell containing the bacteria; the transmission of the current resulting in electrolysis in the vicinity of the electrodes and deviation of the pH levels from bulk values within the cell containing the bacteria.

2. A method for concentration and separation of bacteria as recited in claim 1 wherein the cell provides a specified level of pH; and the created gradient of pH provides an increase or decrease of pH stimulating the bacteria to move along the pH gradient.

3. A method for concentration and separation of bacteria as recited in claim 1 wherein providing a cell containing the bacteria comprises providing a microchannel containing the bacteria.

4. A method for concentration and separation of bacteria as recited in claim 3 wherein providing a microchannel containing the bacteria comprises providing a pair of parallel, spaced-apart glass plates.

5. A method for concentration and separation of bacteria as recited in claim 3 wherein providing a pair of electrodes coupled to said cell containing the bacteria comprises providing a pair of electrodes extending between a pair of parallel, spaced-apart glass plates defining said microchannel.

6. A method for concentration and separation of bacteria as recited in claim 1 wherein providing a cell containing the bacteria includes providing a film containing the bacteria.

7. A method for concentration and separation of bacteria as recited in claim 6 wherein providing a film containing the bacteria includes providing a plurality of support fibers coupled between said film and a frame support.

8. A method for concentration and separation of bacteria as recited in claim 7 wherein said frame support is movable for stretching said film.

9. A method for concentration and separation of bacteria as recited in claim 7 wherein providing a pair of electrodes coupled to said cell containing the bacteria includes providing a pair of electrically conducting wires coupled to said film.

10. A method for concentration and separation of bacteria as recited in claim 1 wherein providing a pair of electrodes coupled to said cell containing the bacteria includes providing a pair of electrically conducting wires coupled to said cell.

11. A method for concentration and separation of bacteria as recited in claim 1 wherein creating a gradient of pH by transmitting an electric current through said cell containing the bacteria includes providing a power supply coupled to said electrodes, said power supply applying a voltage of approximately 2 Volts.

12. A method for concentration and separation of bacteria as recited in claim 1 wherein creating a gradient of pH by transmitting an electric current through said cell containing the bacteria includes providing a power supply coupled to said electrodes, said power supply providing a current in a range of approximately 10-30 μA (10-30 micro-Amperes).

13. A method for concentration and separation of bacteria as recited in claim 1 wherein creating a gradient of pH by transmitting an electric current through said cell containing the bacteria includes generating electrolysis proximate to said pair of electrodes by an applied voltage of approximately 2 Volts to said pair of electrodes for creating the gradient of pH.

14. A method for concentration and separation of bacteria as recited in claim 1 wherein the bacteria includes swimming flagellated bacteria including E. coli, and Bacillus subtilis.

15. Apparatus for concentration and separation of bacteria comprising:
a cell containing the bacteria;
a pair of electrodes coupled to said cell containing the bacteria; and
an electric current transmitted through said cell containing the bacteria for creating a gradient of pH; the transmitted electric current resulting in electrolysis in the vicinity of the electrodes and deviation of the pH levels from bulk values within the cell containing the bacteria.

16. Apparatus for concentration and separation of bacteria as recited in claim 1 wherein said cell containing the bacteria includes a microchannel.

17. Apparatus for concentration and separation of bacteria as recited in claim 1 wherein said cell containing the bacteria includes a thin film.

18. Apparatus for concentration and separation of bacteria as recited in claim 1 wherein said pair of electrodes coupled to said cell containing the bacteria includes a pair of platinum wires.

19. Apparatus for concentration and separation of bacteria as recited in claim 1 wherein said cell containing the bacteria includes a thin film and includes a frame support coupled to said thin film and said frame support being movable for stretching said film.

20. Apparatus for concentration and separation of bacteria as recited in claim 1 wherein said cell containing the bacteria includes a thin film and includes a pair of dielectric fibers coupled to said thin film.
